(19) United States
(12) Patent Application Publication
(10) Pub. No.: US 2025/0144173 A1
(43) Pub. Date: May 8, 2025

(54) GFRALPHA1-CONTAINING NEURITE OUTGROWTH PROMOTER AND PHARMACEUTICAL COMPOSITION FOR INDUCING NERVE REGENERATION

(71) Applicant: NATIONAL UNIVERSITY CORPORATION HOKKAIDO UNIVERSITY, Sapporo-shi (JP)

(72) Inventors: Ken KADOYA, Sapporo-shi (JP); Tomoaki SUZUKI, Sapporo-shi (JP); Takeshi ENDO, Sapporo-shi (JP)

(73) Assignee: NATIONAL UNIVERSITY CORPORATION HOKKAIDO UNIVERSITY, Sapporo-shi (JP)

(21) Appl. No.: 18/259,348

(22) PCT Filed: Dec. 27, 2021
(86) PCT No.: PCT/JP2021/048612

§ 371 Date: Dec. 6, 2023

(30) Foreign Application Priority Data
Dec. 27, 2020 (JP) 2020-218091
May 27, 2021 (JP) 2021-089040

(51) Int. Cl. A61K 38/17; A61P 25/02; C07K 14/71

(52) U.S. Cl. CPC A61K 38/179; A61P 25/02; C07K 14/71; C07K 2319/30

(57) ABSTRACT

The present invention provides a neurite outgrowth promoter for promoting neurite growth on nerve cells of a postnatal mammalian individual for use in a condition in which an effective amount of a glial cell-derived neurotrophic factor (GDNF) is not present, the neurite outgrowth promoter containing at least one protein selected from the group consisting of: GDNF family receptor α1 (GFRα1): GFRα1 mutants having an amino acid sequence that has at least 90% sequence identity with the amino acid sequence of GFRα1, the GFRα1 mutants having neurite outgrowth activity; fusion proteins that have neurite outgrowth activity and that are obtained by fusing GFRα1 or a GFRα1 mutant with another peptide; and chemically modified proteins that have neurite outgrowth activity and that are obtained by chemically modifying one or a plurality of amino acid residues in GFRα1, a GFRα1 mutant, or a fusion protein. The present invention also provides a pharmaceutical composition for inducing nerve regeneration.

### FIELD

The present invention relates to a neurite outgrowth-promoting agent containing GFRα1 for use under a condition in which an effective amount of GDNF is not present, and a pharmaceutical composition for inducing nerve regeneration.

### BACKGROUND

Treatments currently performed for peripheral nerve injuries due to trauma or other causes include follow-up, suturing, and reconstruction using autologous nerves or nerve regeneration-inducing tubes (artificial nerves). Compared with central nerves, peripheral nerves regenerate vigorously, but clinical outcomes are particularly poor in cases of proximal injury, severe injury, and reconstruction because the amount and rate of axon regeneration is not sufficient.

Autologous nerve grafting is the gold standard for peripheral nerve reconstruction but it involves a problem in that healthy autologous nerves outside the injury site must be collected for grafting, so the development of new methods for peripheral nerve regeneration that do not sacrifice autologous nerves is desired. Currently, there is no treatment that has been clinically proven to promote nerve regeneration, and more effective nerve regeneration treatments are needed.

In peripheral nerve regeneration treatments, it is important to promote axon regeneration. Glial cell line-derived neurotrophic factor (GDNF) is one of the neurotrophic factors involved in development and maintenance of neural tissues. GDNF is known to bind to GFRα1 (GDNF family receptor α-1), which is a glycosylphosphatidylisitol (GPI)-anchored receptor, and exhibits an effect on neurons such as an effect of promoting neurite outgrowth, through transmembrane receptor tyrosine kinase RET and neural cell adhesion molecule (NCAM). GFRα1 is also known to act at a distant site, as a soluble factor together with GDNF in the form of soluble GFRα1 in which the GPI anchor is cleaved (Non Patent Literature 1).

Regarding the bioactivity of GFRα1, Non Patent Literature 2 reported that dorsal root ganglion (DRG) neurons isolated from rat embryos showed neurite outgrowth when cultured on GFRα1-coated plates, whereas neurite shortening was observed when cultured in culture media containing GFRα1, suggesting that immobilized GFRα1 and free GFRα1 may have opposing effects on developing neurons.

### CITATION LIST

### Non Patent Literature

Non Patent Literature 1: Ibanez C. F., Biochem Biophys Res Commun. 396(1):24-7, 2010.
Non Patent Literature 2: Mikaels-Edman A. et al., Dev Dyn. 227(1):27-34, 2003.

### SUMMARY

The inventors of the present invention have found that GFRα1 alone promotes neurite outgrowth in neurons of postnatal mammalian individuals even in the absence of GDNF, and that the neurite outgrowth-promoting activity of GFRα1 is stronger than that of GDNF and the activity is further enhanced by inhibiting GDNF function.

The present disclosure provides the followings.

Item 1. A neurite outgrowth-promoting agent for neurons of postnatal mammalian individuals for use under a condition in which an effective amount of GDNF is not present, the neurite outgrowth-promoting agent including at least one protein selected from the group consisting of the following a) to d):
a) GFRα1,
b) a GFRα1 mutant having an amino acid sequence having at least 90% sequence identity with an amino acid sequence of GFRα1, and having neurite outgrowth activity,
c) a fusion protein having neurite outgrowth activity, formed by fusing GFRα1 or a GFRα1 mutant with other peptide, and
d) a chemically modified protein having neurite outgrowth activity, formed by chemically modifying one or more amino acid residues in GFRα1, a GFRα1 mutant, or a fusion protein.

Item 2. A neurite outgrowth-promoting agent for neurons of postnatal mammalian individuals, the neurite outgrowth-promoting agent including at least one protein selected from the group consisting of the following a) to d), and an antibody that inhibits GDNF function or a nucleic acid that suppresses GDNF expression:
a) GFRα1,
b) a GFRα1 mutant having an amino acid sequence having at least 90% sequence identity with an amino acid sequence of GFRα1, and having neurite outgrowth activity,
c) a fusion protein having neurite outgrowth activity, formed by fusing GFRα1 or a GFRα1 mutant with other peptide, and
d) a chemically modified protein having neurite outgrowth activity, formed by chemically modifying one or more amino acid residues in GFRα1, a GFRα1 mutant, or a fusion protein.

Item 3. A neurite outgrowth-promoting agent for neurons of postnatal mammalian individuals, the neurite outgrowth-promoting agent including at least one protein selected from the group consisting of the following a) to d):
a) GFRα1,
b) a GFRα1 mutant having an amino acid sequence having at least 90% sequence identity with an amino acid sequence of GFRα1, and having neurite outgrowth activity,
c) a fusion protein having neurite outgrowth activity, formed by fusing GFRα1 or a GFRα1 mutant with other peptide, and
d) a chemically modified protein having neurite outgrowth activity, formed by chemically modifying one or more amino acid residues in GFRα1, a GFRα1 mutant, or a fusion protein, and
the neurite outgrowth-promoting agent being for use such that, in the surrounding environment of the neurons, the concentration of the at least one protein is in excess of the concentration of GDNF.

Item 4. The agent according to any one of items 1 to 3, wherein the amino acid sequence of GFRα1 is the amino acid sequence set forth in SEQ ID NO: 1, SEQ ID NO: 2, or SEQ ID NO: 3.

Item 5. The agent according to any one of items 1 to 4, wherein a carrier retains the at least one protein.

Item 6. The agent according to any one of items 1 to 4, wherein the at least one protein is included in a free state.

Item 7. A pharmaceutical composition for inducing nerve regeneration, including the agent according to any one of items 1 to 6.

Item 8. A pharmaceutical composition for inducing nerve regeneration for use in treatment of nerve injury, the pharmaceutical composition including at least one protein selected from the group consisting of the following a) to d):
a) GFRα1,
b) a GFRα1 mutant having an amino acid sequence having at least 90% sequence identity with an amino acid sequence of GFRα1, and having neurite outgrowth activity,
c) a fusion protein having neurite outgrowth activity, formed by fusing GFRα1 or a GFRα1 mutant with other peptide, and
d) a chemically modified protein having neurite outgrowth activity, formed by chemically modifying one or more amino acid residues in GFRα1, a GFRα1 mutant, or a fusion protein, and
the treatment including use of an antibody that inhibits GDNF function or a nucleic acid that suppresses GDNF expression.

Item 9. The pharmaceutical composition according to item 7 or 8, wherein the pharmaceutical composition is in a form in which a biocompatible medical material retains the at least one protein.

Item 10. A drug for treatment of nerve injury, including at least one protein selected from the group consisting of the following a) to d) in combination with an antibody that inhibits GDNF function or a nucleic acid that suppresses GDNF expression:
a) GFRα1,
b) a GFRα1 mutant having an amino acid sequence having at least 90% sequence identity with an amino acid sequence of GFRα1, and having neurite outgrowth activity,
c) a fusion protein having neurite outgrowth activity, formed by fusing GFRα1 or a GFRα1 mutant with other peptide, and
d) a chemically modified protein having neurite outgrowth activity, formed by chemically modifying one or more amino acid residues in GFRα1, a GFRα1 mutant, or a fusion protein.

Item 11. An NCAM stimulating agent for neurons of postnatal mammalian individuals for use under a condition in which an effective amount of GDNF is not present, the NCAM stimulating agent including at least one protein selected from the group consisting of the following a) to d):
a) GFRα1,
b) a GFRα1 mutant having an amino acid sequence having at least 90% sequence identity with an amino acid sequence of GFRα1, and having neurite outgrowth activity,
c) a fusion protein having neurite outgrowth activity, formed by fusing GFRα1 or a GFRα1 mutant with other peptide, and
d) a chemically modified protein having neurite outgrowth activity, formed by chemically modifying one or more amino acid residues in GFRα1, a GFRα1 mutant, or a fusion protein.

Item 12. An NCAM stimulating agent for neurons of postnatal mammalian individuals, the NCAM stimulating agent including at least one protein selected from the group consisting of the following a) to d), and an antibody that inhibits GDNF function or a nucleic acid that suppresses GDNF expression:
a) GFRα1,
b) a GFRα1 mutant having an amino acid sequence having at least 90% sequence identity with an amino acid sequence of GFRα1, and having neurite outgrowth activity,
c) a fusion protein having neurite outgrowth activity, formed by fusing GFRα1 or a GFRα1 mutant with other peptide, and
d) a chemically modified protein having neurite outgrowth activity, formed by chemically modifying one or more amino acid residues in GFRα1, a GFRα1 mutant, or a fusion protein.

Item 13. An NCAM stimulating agent for neurons of postnatal mammalian individuals, the NCAM stimulating agent including at least one protein selected from the group consisting of the following a) to d):
a) GFRα1,
b) a GFRα1 mutant having an amino acid sequence having at least 90% sequence identity with an amino acid sequence of GFRα1, and having neurite outgrowth activity,
c) a fusion protein having neurite outgrowth activity, formed by fusing GFRα1 or a GFRα1 mutant with other peptide, and
d) a chemically modified protein having neurite outgrowth activity, formed by chemically modifying one or more amino acid residues in GFRα1, a GFRα1 mutant, or a fusion protein, and
the NCAM stimulating agent being for use such that, in the surrounding environment of the neurons, the concentration of the at least one protein is in excess of the concentration of GDNF.

Item 14. The agent according to any one of items 11 to 13, wherein the amino acid sequence of GFRα1 is the amino acid sequence set forth in SEQ ID NO: 1, SEQ ID NO: 2, or SEQ ID NO: 3.

Item 15. The agent according to any one of items 11 to 14, wherein a carrier retains the at least one protein.

Item 16. The agent according to any one of items 11 to 14, wherein the at least one protein is included in a free state.

According to the present disclosure, effective nerve regeneration can be achieved.

### BRIEF DESCRIPTION OF DRAWINGS

FIG. 1 is a schematic diagram of a putative molecular mechanism of GFRα1.

FIG. 2 is an image illustrating a representative example of adult rat dorsal root ganglion cells (DRG cells) cultured in a culture medium containing GFRα1-Fc. DRG cells were labeled with anti-pan neurofilament (pNF) antibody.

FIG. 3 includes graphs of the percentage of elongating neurons and the longest neurite length of DRG cells cultured in a culture medium containing GFRα1-Fc.

FIG. 4 is an image illustrating a representative example of DRG cells cultured on a Poly-L-lysine (PLL) and Laminin coated plate with additional coating of GFRα1-Fc. DRG cells were labeled with anti-pNF antibody.

FIG. 5 includes graphs of the percentage of elongating neurons and the longest neurite length of DRG cells cultured on a PLL and Laminin-coated plate with additional coating of GFRα1-Fc.

FIG. 6 includes graphs of the percentage of elongating neurons and the longest neurite length of DRG cells cultured on a PLL and Laminin-coated plate with additional coating of GFRα1-Fc with different concentrations.

FIG. 7 is a diagram illustrating the results of western blotting analysis of lysate of DRG cells cultured on a PLL and Laminin-coated plate with additional coating of GFRα1-Fc.

FIG. 8 includes graphs of the percentage of elongating neurons and the longest neurite length of DRG cells cultured on a PLL and Laminin-coated plate with additional coating of GFRα1-Fc in the presence of NCAM inhibitory antibody.

FIG. 9 includes graphs of the percentage of elongating neurons and the longest neurite length of DRG cells cultured on a PLL and Laminin-coated plate with additional coating of GFRα1-Fc in the presence of integrin 31 inhibitory antibody.

FIG. 10 is an image illustrating a representative example of DRG cells cultured on a PLL and Laminin-coated plate or a PLL-coated plate with additional coating of GFRα1-Fc. DRG cells were labeled with anti-pNF antibody.

FIG. 11 includes graphs of the percentage of elongating neurons and the longest neurite length of DRG cells cultured on a PLL and Laminin-coated plate or a PLL-coated plate with additional coating of GFRα1-Fc.

FIG. 12 is a diagram illustrating the results of western blotting analysis of lysate of DRG cells cultured on a PLL and Laminin-coated plate with additional coating of GFRα1-Fc in the presence of GDNF and/or GDNF inhibitory antibody.

FIG. 13 includes graphs of the relative intensity of each band detected by western blotting illustrated in FIG. 12.

FIG. 14 includes graphs of the percentage of elongating neurons and the longest neurite length of DRG cells cultured on a PLL and Laminin-coated plate with additional coating of GFRα1-Fc in the presence of GDNF inhibitory antibody.

FIG. 15 includes graphs of the percentage of elongating neurons and the longest neurite length of DRG cells cultured on a PLL and Laminin-coated plate with additional coating of GFRα1-Fc in the presence of GDNF (the upper part of FIG. 15), and a conceptual diagram illustrating the localization of GFRα1-Fc and GDNF during culture (the lower part of FIG. 15).

FIG. 16 includes graphs of the percentage of elongating neurons and the longest neurite length of DRG cells cultured on a PLL and Laminin-coated plate with additional coating of GFRα1-Fc in the presence of GDNF and/or GDNF inhibitory antibody.

FIG. 17 includes graphs of the percentage of elongating neurons and the longest neurite length of DRG cells cultured in culture media containing GFRα1-Fc from different origins.

FIG. 18 is an image of a representative example of sciatic nerves of sciatic nerve-injured rats to which GFRα1-Fc was administered locally. The sciatic nerves were labeled with anti-SCG10 antibody.

FIG. 19 is a graph illustrating the relationship between the distance from the injury site and the percentage of axon regeneration for the sciatic nerves of sciatic nerve-injured rats to which GFRα1-Fc was administered locally.

FIG. 20 includes graphs illustrating the response time to pain stimulation in sciatic nerve-injured rats to which GFRα1-Fc was administered locally.

FIG. 21 includes graphs of the weights of anterior tibial muscle and gastrocnemius muscle of sciatic nerve-injured rats to which GFRα1-Fc was administered locally.

FIG. 22 includes graphs of the latency and the amplitude of electrical stimulation in sciatic nerve-injured rats to which GFRα1-Fc was administered locally.

FIG. 23 is an image illustrating a representative example of sciatic nerves of sciatic nerve-injured rats to which GFRα1-Fc or a combination of GFRα1-Fc and GDNF inhibitory antibody was administered. The sciatic nerves were labeled with anti-SCG10 antibody. In the image, white arrows indicate the longest axons.

FIG. 24 includes graphs illustrating the relationship between the distance from the injury site and the percentage of axon regeneration for sciatic nerves of sciatic nerve-injured rats to which GFRα1-Fc or a combination of GFRα1-Fc and GDNF inhibitory antibody was administered locally (the left part of FIG. 24), and illustrating the maximum length of regenerated axon (the right part of FIG. 24).

FIG. 25 includes photographs of a rat sciatic nerve defect before and after implantation of a GFRα1-Fc-coated implant.

FIG. 26 is an image illustrating a representative example of axon regeneration in a sciatic nerve defect in a rat to which the GFRα1-Fc-coated implant was implanted. The sciatic nerves were labeled with anti-SCG10 antibody. In the image, white arrows indicate the longest axons.

FIG. 27 is a graph of the maximum length of regenerated axon in a sciatic nerve defect in a rat to which the GFRα1-Fc-coated implant was implanted.

### DESCRIPTION OF EMBODIMENTS

The following description may be based on representative embodiments or specific examples, but the present invention is not limited to such embodiments or specific examples. The description of proteins and genes will be given with human-derived proteins and genes as examples unless otherwise specified, but proteins and genes used in the present invention are not limited to human-derived ones, and the subject to which the present invention is applied is not limited to humans.

In the present specification, the upper limit value and the lower limit value of each numerical range can be combined as desired. In the present specification, a numerical range represented using “to” or “-” means a range including numerical values at both ends thereof as an upper limit value and a lower limit value, unless otherwise noted.

In one aspect, the present invention provides a neurite outgrowth-promoting agent for neurons of postnatal mammalian individuals for use under a condition in which an effective amount of GDNF is not present, the neurite outgrowth-promoting agent including at least one protein selected from the group consisting of GFRα1, GFRα1 mutants, and GFRα1 derivatives. In one aspect, the present invention also provides a neurite outgrowth-promoting agent for neurons of postnatal mammalian individuals, the neurite outgrowth-promoting agent including the at least one protein and an antibody that inhibits GDNF function or a nucleic acid that suppresses GDNF expression. In one aspect, the present invention further provides a neurite outgrowth-promoting agent for neurons of postnatal mammalian individuals, the neurite outgrowth-promoting agent including the at least one protein, and the neurite outgrowth-promoting agent being for use such that, in the surrounding environment of the neurons, the concentration of the at least one protein is in excess of the concentration of GDNF.

### Neurons of Postnatal Mammalian Individuals

In the present disclosure, neurons of postnatal mammalian individuals mean neurons present in postnatal mammalian individuals or collected from postnatal mammalian individuals, and are clearly distinguished from neurons of prenatal mammalian individuals such as embryos or fetuses. The neurons of postnatal mammalian individuals may be neurons of the peripheral nervous system or neurons of the central nervous system, and neurons of peripheral nerves are preferred. Neurons of the peripheral nervous system may be neurons of either somatic nervous system (sensory nerves and motor nerves) or autonomic nervous system (sympathetic nerves and parasympathetic nerves).

### GFRα1, GFRα1 Mutant, and GFRα1 Derivative

GFRα1 is a receptor for GDNF and is known to have the function of transducing GDNF signals to cells in a RET-dependent or independent manner. Human GFRα1 (registered as P56159 in the protein sequence database UniProtKB) is translated as a protein composed of 465 (Isoform a) or 460 (Isoform b) amino acid residues with a signal peptide composed of 24 amino acid residues at the N-terminal side and a propeptide composed of 36 amino acid residues on the C-terminal side. This protein undergoes processing and post-translational modifications to become a mature form of GFRα1 with GPI attached to the C terminal. The GPI-attached GFRα1 is anchored to a cell surface and binds to GDNF in a state called GPI-anchored GRFα1 to exhibit its function. On the other hand, when undergoing GPI cleavage, the GPI-anchored GFRα1 becomes unattached to a cell membrane, which state is called soluble GFRα1, and binds to GDNF to exhibit its function away from its original site.

In the present disclosure, GFRα1 can be used as a neurite outgrowth-promoting agent for neurons of postnatal mammalian individuals, under a condition in which an effective amount of GDNF is not present, or under a condition of coexistence with an antibody that inhibits GDNF function or a nucleic acid that suppresses GDNF expression, or under a condition in which the concentration of GFRα1 is in excess of the concentration of GDNF. GFRα1 may be derived from the same animal species as the neurons to which it is applied, or may be derived from a different animal species. For example, rat GFRα1 may be used for rat neurons, or GFRα1 from other animal species, for example, mouse or human GFRα1 may be used for rat neurons. In the present disclosure, in addition to GFRα1, a GFRα1 mutant and a GFRα1 derivative that retain the neurite outgrowth-promoting activity of GFRα1 can also be used as neurite outgrowth-promoting agents.

The GFRα1 mutant in the present disclosure is a protein in which the amino acid sequence of GFRα1 is modified while retaining the neurite outgrowth-promoting activity of GFRα1. One example of such an amino acid sequence is an amino acid sequence having at least 70% or more, preferably 80% or more, more preferably 90% or more, even more preferably 95% or more, and particularly preferably 97%, 98% or 99% or more sequence identity with the amino acid sequence of wild-type GFRα1, for example, GFRα1 having the amino acid sequence set forth in SEQ ID NO: 1 (Isoform a) registered as NCBI Reference Sequence: NP_005255.1, SEQ ID NO: 2 (Isoform b) registered as NP_665736.1, or SEQ ID NO: 3 (Isoform c) registered as NP_001335028.1. Another example is an amino acid sequence in which up to 40 amino acid residues, for example, 1 to 40, 1 to 30, 1 to 25, 1 to 20, 1 to 15, 1 to 10, or 1 to 5 amino acid residues are deleted or substituted in a natural amino acid sequence of GFRα1. The natural amino acid sequence is, for example, the amino acid sequence set forth in SEQ ID NO: 1, 2, or 3.

The substitution is preferably conservative substitution, and examples thereof include substitutions between amino acids, such as glycine (Gly) and proline (Pro), glycine and alanine (Ala) or valine (Val), leucine (Leu) and isoleucine (lie), glutamic acid (Glu) and glutamine (Gln), aspartic acid (Asp) and asparagine (Asn), cysteine (Cys) and threonine (Thr), threonine and serine (Ser) or alanine, and lysine (Lys) and arginine (Arg).

Amino acid sequence identity is represented by the ratio of identical amino acid residues to the alignment length, and the alignment of the two compared amino acid sequences is performed according to standard methods such that the number of identical amino acid residues is maximized. Sequence identity can be determined by any method known to those skilled in the art, for example, using a sequence comparison program such as BLAST.

A polypeptide fragment of GFRα1 having neurite outgrowth-promoting activity is also encompassed in the GFRα1 mutant. The polypeptide fragment of GFRα1 preferably consists of or includes 19th to 429th amino acid residues in SEQ ID NO: 1.

The GFRα1 derivative in the present disclosure is a protein obtained by attaching other substance(s) while retaining the neurite outgrowth-promoting activity of GFRα1. Preferred examples include a fusion protein having neurite outgrowth activity, formed by fusing GFRα1 or a GFRα1 mutant with other peptide(s), and a chemically modified protein having neurite outgrowth activity, formed by chemically modifying one or more amino acid residues in GFRα1, a GFRα1 mutant, or a fusion protein.

Examples of other peptide(s) in the fusion protein include the Fc region of human IgG (Fc protein), His tag, GST tag, HA tag, FLAG tag, and the like. Examples of the chemical modification include acylation, prenylation, acetylation, phosphorylation, glycosylation, and PEGylation.

GFRα1 does not require GPI attachment to exhibit neurite outgrowth-promoting activity. Therefore, GFRα1, the GFRα1 mutant, and the GFRα1 derivative used in the present disclosure may have or may not have GPI attached, and the use of GFRα1 without GPI attached is preferred.

Instead of the above GFRα1, GFRα1 mutant, or GFRα1 derivative, a nucleic acid encoding each of them can also be used as the neurite outgrowth-promoting agent.

### Preparation of GFRα1, GFRα1 Mutant, and GFRα1 Derivative

GFRα1, the GFRα1 mutant, and the GFRα1 derivative (hereinafter referred to as GFRα1 and variants thereof. As used herein, the terms “GFRα1 and variants thereof” and “GFRα1 or variants thereof” can be replaced with “at least one protein selected from the group consisting of GFRα1, GFRα1 mutants, and GFRα1 derivatives”, depending on the context.) can be produced by genetic engineering production methods that include introducing expression vectors containing nucleic acids encoding these into appropriate host cells, such as E. coli or other microorganisms, insect cells, or animal cells, and then expressing them. The operations in the genetic engineering production methods, including preparation of nucleic acids, types of host cells and methods of introducing nucleic acids, protein expression and purification, can be performed by methods known or well known to those skilled in the art, based on the instructions in experimental operation manuals that explain various genetic engineering operations in detail. In one aspect, the present invention also provides a nucleic acid encoding each of GFRα1 and variants thereof, an expression vector including the nucleic acid, and a host cell transformed with the nucleic acid.

A cell-free synthesis method using the nucleic acid encoding each of GFRα1 and variants thereof is also one of the genetic engineering production methods for GFRα1 or mutants thereof. Examples of a cell-free protein synthesis system include systems using extracts of cells such as E. coli, wheat germ, yeast, rabbit reticulocytes, insect cells, and cultured mammalian cells, and reconstituted systems composed by combining factors necessary for protein synthesis.

Furthermore, GFRα1 and variants thereof can be produced by organic chemical synthetic methods such as the Fmoc method (fluorenylmethyloxycarbonyl method) or the tBoc method (t-butyloxycarbonyl method), using amino acids modified with various protecting groups as raw materials, and preferably produced by introducing the above nucleic acid, especially DNA in which the nucleic acid is incorporated in a expression vector, into a suitable expression system using a suitable host cell selected from prokaryotes or eukaryotes.

Use of GFRα1 and Variant Thereof Under Condition in which Effective Amount of GDNF is not Present

GFRα1 and variants thereof can be used to promote neurite outgrowth for neurons of postnatal mammalian individuals under a condition in which an effective amount of GDNF is not present. The term “an effective amount of GDNF is not present” means that GDNF is not present in such an amount that can exhibit neurite outgrowth-promoting activity in the surrounding environment of neurons for which neurite outgrowth is desired. Even though a certain concentration of GDNF is present in the surrounding environment of neurons for which neurite outgrowth is desired, if no promotion of neurite outgrowth is observed, an effective amount of GDNF is not present in the surrounding environment.

For example, the central nervous system is under a condition in which an effective amount of GDNF is not present, because the central nervous system is deficient in neurotrophic factors such as GDNF. The peripheral nervous system of the elderly is also under a condition in which an effective amount of GDNF is not present, because the function of Schwann cells is reduced in the elderly and their ability to produce GDNF is diminished.

Furthermore, “a condition in which an effective amount of GDNF is not present” encompasses a condition in which GDNF is virtually not present, specifically, a condition in which the concentration of GDNF in the surrounding environment of neurons is below the limit of detection by immunoassay using a specific antibody for GDNF. For example, a medical material for inducing nerve regeneration, which is a cell-free scaffold, is under a condition in which GDNF is virtually not present, unless special treatment to impart GDNF is performed.

The absence of an effective amount of GDNF in an environment can be confirmed by observing that neurite outgrowth of neurons does not occur unless a GDNF-independent GFRα1 putative molecular mechanism (left side of FIG. 1) described later is activated in the environment. For example, an effective amount of GDNF is not present in an environment when neurite outgrowth of neurons is observed in the presence of a ligand for integrin α7β1 such as laminin but neurite outgrowth of neurons is not observed when the ligand is deficient. For example, an effective amount of GDNF is not present in an environment when neurite outgrowth is observed in normal neurons but neurite outgrowth is not observed in neurons lacking NCAM or integrin α7β1.

Use in Combination with GDNF-Inhibitory Antibody or GDNF Expression-Suppressing Nucleic Acid

GFRα1 and variants thereof can be used to promote neurite outgrowth for neurons of postnatal mammalian individuals, under coexistence with an antibody that inhibits GDNF function or a nucleic acid that suppresses GDNF expression. The coexistence with the antibody that inhibits GDNF function or a nucleic acid that suppresses GDNF expression results in the suppression of GDNF expression or function, which makes the surrounding environment of the neurons a condition in which an effective amount of GDNF is not present, thereby enabling GFRα1 and variants thereof to exhibit neurite outgrowth activity.

The antibody that inhibits GDNF function is a specific antibody for GDNF or a derivative thereof. The specific antibody for GDNF can also be represented as an antibody that binds preferentially to GDNF over non-target proteins, or an antibody having high binding affinity for GDNF. The specific antibody for GDNF can bind to GDNF with affinity at least twice stronger, preferably at least 10 times stronger, more preferably at least 20 times stronger, and most preferably at least 100 times stronger than affinity to non-target proteins.

The specific antibody for GDNF can originate from any species including, for example, mice, rats, sharks, rabbits, pigs, hamsters, camels, llamas, goats, or humans. The specific antibody can be of any class (for example, IgG, IgE, IgM, IgD, or IgA) and subclass of immunoglobulin molecules, and IgG is preferred.

In the present disclosure, the specific antibody for GDNF can be a polyclonal antibody or a monoclonal antibody, and a monoclonal antibody is preferred. The specific antibody may be a chimeric antibody, a humanized antibody, or a human antibody.

A derivative of the specific antibody for GDNF may be an antigen-binding fragment derived from the specific antibody for GDNF, which is defined as a partial fragment of an antibody having the ability to bind specifically to the antigen. Examples of such fragments can include, but not limited to, fragment of antigen binding (Fab), Fab′, F(ab′)2, single chain Fv, disulfide stabilized Fv, and peptides containing CDR.

The specific antibody for GDNF and a derivative thereof can be produced using methods known to those skilled in the art. For example, they can be produced by preparing GDNF by genetic recombination techniques based on the amino acid sequence of human GDNF (for example, registered as NCBI Reference Sequence: NP_000505.1) or the nucleotide sequence of DNA encoding it (for example, registered as NCBI Reference Sequence: NM_000514), immunizing an appropriate animal with the GDNF as an antigen, and then fusing B cells of the animal with myeloma cells to obtain hybridomas. For the hybridoma method, see, for example, Meyaard et al. (1997) Immunity 7:283-290; Wright et al. (2000) Immunity 13:233-242; Kaithamana et al. (1999) J. Immunol. 163:5157-5164. It is also possible to use commercially available anti-GDNF antibodies, such as anti-human/rat GDNF antibody (AF-212-SP, R&D Systems), anti-human GDNF antibody (A16099G, BioLegend), antibodies including, as CDR sequences, amino acid sequences identical to or with 90% or more sequence identity with the CDR sequences of these antibodies, and antibodies including, as the heavy chain variable region and the light chain variable region, amino acid sequences identical to or with 90% or more sequence identity with the amino acid sequences of the heavy chain variable region and the light chain variable region of these antibodies.

In the present disclosure, an aptamer that inhibits GDNF function can also be used instead of the antibody that inhibits GDNF function. Aptamers are molecules that have the ability to bind specifically to target substances with their three-dimensional structure. Aptamers composed of nucleic acids are called nucleic acid aptamers, and aptamers composed of amino acids are called peptide aptamers. In the present invention, either nucleic acid aptamers or peptide aptamers can be used.

The nucleic acid aptamer used in the present disclosure can be deoxyribonucleic acid (DNA), ribonucleic acid (RNA), or chimeric nucleic acid including both deoxyribonucleotide and ribonucleotide as constituent units. The nucleic acid aptamer can be produced by methods known to those skilled in the art, such as the systematic evolution of ligands by exponential enrichment (SELEX) method. The peptide aptamer can be produced by methods known to those skilled in the art, such as phage display or cell surface display.

The nucleic acid that suppresses GDNF expression encompasses a nucleic acid capable of suppressing transcription of mRNA from GDNF gene (GDNF mRNA), a nucleic acid capable of degrading GDNF mRNA, and a nucleic acid capable of suppressing protein translation from GDNF mRNA. Examples include an antisense nucleic acid or a nucleic acid causing RNA interference, such as siRNA, shRNA, or miRNA, which can be designed and produced by those skilled in the art based on the nucleotide sequence of GDNF gene or the nucleotide sequence of GDNF mRNA.

In addition, DNA capable of inducing transcription of the antisense nucleic acid or the nucleic acid causing RNA interference when positioned under the control of an appropriate expression promoter, and a nucleic acid in which a part of the nucleotide sequence of the antisense nucleic acid or the nucleic acid causing RNA interference is modified to enhance the stability against degradation by nucleases are also encompassed in the nucleic acid that suppresses GDNF expression in the present disclosure, as a nucleic acid functionally equivalent to the antisense nucleic acid or the nucleic acid causing RNA interference. Examples of the modification to improve the stability against degradation by nucleases include 2′O-methylation, 2′-fluorination, and 4′-thiolation.

Furthermore, a chimeric RNA in which a portion of ribonucleotides constituting RNA that suppresses GDNF expression is substituted by corresponding deoxyribonucleotide(s) or nucleotide analog(s) is also encompassed in the nucleic acid that suppresses GDNF expression. Examples of the nucleotide analog include 5-position-modified uridine or cytidine, such as 5-(2-amino)propyluridine and 5-bromouridine; 8-position-modified adenosine or guanosine, such as 8-bromoguanosine; deaza-nucleotides, such as 7-deaza-adenosine; O- or N-alkylated nucleotides, such as N6-methyladenosine. The type or number of bases to be modified or substituted is not limited as long as the ability to suppress the expression of the target molecule is not lost.

The nucleic acid that suppresses GDNF expression can be artificially synthesized using genetic recombination or chemical synthesis techniques. Genetic recombination methods, chemical synthesis methods for nucleic acids, as well as techniques of synthesizing non-natural-type bases or techniques of synthesizing nucleic acids containing these are well known to those skilled in the art.

### Use of GFRα1 and Variant Thereof in Excess of GDNF

GFRα1 and variants thereof can be used in such a way that their concentration exceeds that of GDNF in the surrounding environment of neurons of postnatal mammalian individuals, in order to promote neurite outgrowth for the neurons. When there is an excess amount of GFRα1 beyond what GDNF can bind in the surrounding environment of neurons, it results in GFRα1 not bound to GDNF. This unbound GFRα1 is presumed to exhibit neurite outgrowth activity independent of GDNF, similar to the condition in which an effective amount of GDNF is not present.

The concentration of GFRα1 and variants hereof in excess of the concentration of GDNF can be determined experimentally. When two or more proteins selected from the group consisting of GFRα1 and variants thereof are used, it is sufficient that the total concentration of the two or more proteins is in excess of the GDNF concentration, and the concentration of each individual protein need not be in excess.

### Neurite Outgrowth-Promoting Agent

As described above, GFRα1 and variants thereof can be used each alone or in combination of two or more proteins among them, as a neurite outgrowth-promoting agent. In certain embodiments, GFRα1 and variants thereof can be further combined with an antibody that inhibits GDNF function or a nucleic acid that suppresses GDNF expression, and used as a neurite outgrowth-promoting agent. The neurite outgrowth-promoting agent can be a composition containing two or more substances.

In embodiments of the neurite outgrowth-promoting agent including a combination of two or more proteins among GFRα1 and variants thereof, and in embodiments of the neurite outgrowth-promoting agent further including an antibody that inhibits GDNF function or a nucleic acid that suppresses GDNF expression in addition to GFRα1 or variants thereof, all the components that constitute the agent may be contained in a single container or in separate containers. The components that constitute the agent may be applied simultaneously or separately to neurons of postnatal mammalian individuals for which neurite outgrowth is desired.

GFRα1 and variants thereof may be used in a state retained in or on a carrier (immobilized state) or may be used in a free state (free state). Examples of the carrier which retains GFRα1 and variants thereof include culture substrates such as well plates and petri dishes commonly used in cell culture, medical materials for inducing nerve regeneration, and particles such as microbeads and liposomes. The method of making a carrier retain GFRα1 and variants thereof is not specifically restricted. For example, GFRα1 and variants thereof may be directly fixed to a carrier, may be fixed through an appropriate linker molecule or an adapter molecule, or may be fixed using a binder or coating material. A carrier may retain GFRα1 and variants thereof by nonspecific adsorption. When the carrier is a gel-like material, GFRα1 and variants thereof may be included in a solution in a gel preparation process, thereby allowing the carrier to retain GFRα1 and variants thereof.

GFRα1 and variants thereof in a free state can be used, for example, in a liquid such as a culture medium for cell culture, buffer solution, physiological saline, or phosphate-buffered saline.

As described later, the neurite outgrowth-promoting activity of GFRα1 under a condition in which an effective amount of GDNF is not present requires laminin, which is a ligand for integrin α7β1. When GFRα1 and variants thereof are used as neurite outgrowth-promoting agents in vivo, laminin does not have to be externally added because it is present around neurons as an extracellular matrix. On the other hand, when GFRα1 and variants thereof are used as neurite outgrowth-promoting agents under a condition in which laminin is not originally present, for example, when used in vitro, laminin needs to be introduced in addition to GFRα1 and variants thereof.

### Pharmaceutical Composition

The neurite outgrowth-promoting agent can be used either in vitro or in vivo. The neurite outgrowth-promoting agent used in vivo can promote axon regeneration of damaged neurons present in a living body, leading to restoration of nerve function. Thus, the neurite outgrowth-promoting agent can be used for treatment of nerve injury as a pharmaceutical composition for inducing nerve regeneration.

As described above, the central nervous system and the peripheral nervous system in the elderly are under a condition in which an effective amount of GDNF is not present. Thus, the neurite outgrowth-promoting agent for neurons of postnatal mammalian individuals for use under a condition in which an effective amount of GDNF is not present can be used as a pharmaceutical composition for inducing nerve regeneration for treatment of injury in the central nerve system and for treatment of nerve injury in the elderly.

As used herein, the term “treatment” encompasses all types of medically acceptable therapeutic interventions aimed at cure or temporary remission of a disease or a condition. Thus, treatment of nerve injury encompasses medically acceptable interventions for a variety of purposes, including improvement of functional disorder caused by nerve injury, delay or stop of the progression of functional disorder, and prevention of the onset.

Nerve injury may be injury either in the peripheral nerve system or central nerve system, and may be open injury caused by wounding, or closed injury such as compression, subcutaneous fracture, contusion, traction injury, electric shock injury, drug injection, and radiation damage. The pharmaceutical composition can be used for nerve injury where axonal rupture has occurred, and preferably used for severe nerve injury where spontaneous recovery is not expected.

In the present disclosure, treatment of nerve injury also encompasses inducing regeneration of injured axons and thereby treating a disease associated with axonal injury. Examples of the disease associated with axonal injury include traumatic diseases (brain trauma, spinal cord injury, optic nerve injury, facial nerve injury, peripheral neuropathy), infarction (cerebral infarction, spinal cord infarction), inflammatory nerve diseases (multiple sclerosis), neurodegenerative disorders (Parkinson's disease, Alzheimer's disease, ALS), metabolic neuropathy, drug and toxic neuropathy, and hereditary neuropathy.

The pharmaceutical composition is administered to subjects with nerve injury, for example, postnatal mammalian individuals such as rodents including mice, rats, hamsters, and guinea pigs, primates including humans, chimpanzees, and rhesus monkeys, domestic animals including pigs, cattle, goats, horses, and sheep, and pet animals including dogs and cats. Preferred subjects are humans.

The pharmaceutical composition includes an effective amount of GFRα1 or variants thereof for treatment of nerve injury and, in certain embodiments, further includes an antibody that inhibits GDNF function or a nucleic acid that suppresses GDNF expression. Each effective amount is determined as appropriate according to usage, subject age, gender, weight, site and degree of nerve injury, and other factors.

The pharmaceutical composition can include other medical agents for treatment of nerve injury, or pharmaceutically acceptable additives, such as a buffer, a stabilizer, a preservative, and an excipient. The pharmaceutically acceptable additives are well known to those skilled in the art and can be selected and used by those skilled in the art within the normal practice ability.

The dosage form of the pharmaceutical composition is not limited, and parenteral preparation such as injection is preferred. The administration route of the pharmaceutical composition is not limited as long as an active ingredient can be delivered to the nerve injury site, and local administration at or near the nerve injury site is preferred. The pharmaceutical composition may also be in the form in which an effective amount of GFRα1 or variants thereof is retained in or on a substrate such as a biocompatible medical material.

In one embodiment, GFRα1 or variants thereof and an antibody that inhibits GDNF function or a nucleic acid that suppresses GDNF expression can be used as a combination drug for treatment of nerve injury. The term “combination drug” means a combination of two or more drugs intended to be administered together or separately, simultaneously or sequentially, to a subject that needs treatment of nerve injury. Examples of a manner of administration intended include administration of a preparation in which GFRα1 or variants thereof and an antibody that inhibits GDNF function or a nucleic acid that suppresses GDNF expression are contained in a single formulation, and administration of GFRα1 or variants thereof and an antibody that inhibits GDNF function or a nucleic acid that suppresses GDNF expression that are formulated separately. When they are formulated and administered separately, the order and timing of their administration are not limited, and they may be administered simultaneously or may be administered at different times or on different days with an intervening period.

Each individual pharmaceutical composition used in the combination drug can be referred to, respectively, as: a pharmaceutical composition including GFRα1 or variants thereof for treatment of nerve injury, wherein the treatment includes use of an antibody that inhibits GDNF function or a nucleic acid that suppresses GDNF expression; and a pharmaceutical composition including an antibody that inhibits GDNF function or a nucleic acid that suppresses GDNF expression for treatment of nerve injury, wherein the treatment includes use of GFRα1 or variants thereof.

In one aspect, the present invention provides a method for treating nerve injury, including administering a pharmaceutical composition containing an effective amount of at least one protein selected from the group consisting of GFRα1, GFRα1 mutants, and GFRα1 derivatives, to a postnatal mammalian individual in which an effective amount of GDNF is not present at or near a nerve injury site. In one aspect, the present invention also provides a method for treating nerve injury, including administering a pharmaceutical composition containing an effective amount of the at least one protein and an antibody that inhibits GDNF function or a nucleic acid that suppresses GDNF expression, to a postnatal mammalian individual. In one aspect, the present invention further provides a method for treating nerve injury, including administering a pharmaceutical composition containing an effective amount of the at least one protein and administering a pharmaceutical composition containing an antibody that inhibits GDNF function or a nucleic acid that suppresses GDNF expression, to a postnatal mammalian individual. In one aspect, the present invention additionally provides a method for treating nerve injury, including administering a pharmaceutical composition containing the at least one protein to a postnatal mammalian individual such that, at or near a nerve injury site, the concentration of the at least one protein is in excess of the concentration of GDNF concentration.

In one aspect, the present invention provides at least one protein selected from the group consisting of GFRα1, GFRα1 mutants, and GFRα1 derivatives, for use in inducing nerve regeneration in a postnatal mammalian individual in which an effective amount of GDNF is not present at or near a nerve injury site, and for use in treating nerve injury in the individual. In one aspect, the present invention also provides the at least one protein and an antibody that inhibits GDNF function or a nucleic acid that suppresses GDNF expression, for use in inducing nerve regeneration in a postnatal mammalian individual, and for use in treating nerve injury in the individual. In one aspect, the present invention additionally provides the at least one protein for use in inducing nerve regeneration in a postnatal mammalian individual, and for use in treating nerve injury in the individual, wherein the at least one protein is used such that, at or near a nerve injury site, the concentration of the at least one protein is in excess of the concentration of GDNF.

In one aspect, the present invention provides use of at least one protein selected from the group consisting of GFRα1, GFRα1 mutants, and GFRα1 derivatives, in production of a pharmaceutical composition for inducing nerve regeneration for use in a postnatal mammalian individual in which an effective amount of GDNF is not present at or near a nerve injury site. In one aspect, the present invention also provides use of the at least one protein and an antibody that inhibits GDNF function or a nucleic acid that suppresses GDNF expression, in production of a pharmaceutical composition for inducing nerve regeneration for use in a postnatal mammalian individual. In one aspect, the present invention additionally provides use of the at least one protein, in production of a pharmaceutical composition for inducing nerve regeneration for use in a postnatal mammalian individual, wherein the pharmaceutical composition is administered such that, at or near a nerve injury site, the concentration of the at least one protein is in excess of the concentration of GDNF.

### Medical Material for Inducing Nerve Regeneration

GFRα1 and variants thereof can be retained in or on a biocompatible medical material and implanted to a nerve injury site in a living body, and thereby can also be used as a medical material for inducing nerve regeneration.

Examples of the biocompatible medical material include polymer compounds such as polyfluoroethylene and polystyrene, inorganic compounds such as silica, and biodegradable polymers, and biodegradable polymers are preferred. Examples of the biodegradable polymers include synthetic polymer materials including polyglycolic acid, polylactic acid, polyethylene glycol, polycaprolactone, polydioxanone, and copolymers thereof, such as poly(lactic-co-glycolic acid); inorganic materials including β-tricalcium phosphate and calcium carbonate; and natural polymer materials including collagen, gelatin, alginic acid, hyaluronic acid, agarose, chitosan, fibrin, fibroin, chitin, cellulose, and silk.

The medical material for inducing nerve regeneration retains an effective amount of GFRα1 or variants thereof for treatment of nerve injury and, in certain embodiments, further retains an antibody that inhibits GDNF function or a nucleic acid that suppresses GDNF expression. Each effective amount is determined as appropriate according to subject age, gender, weight, site and degree of nerve injury, and other factors. The method of making a biocompatible medical material retain GFRα1 and variants thereof is as described above in relation to the neurite outgrowth-promoting agent.

The medical material for inducing nerve regeneration can be used for treatment of nerve injury. The nerve injury treated by the medical material for inducing nerve regeneration, the subject to which the medical material for inducing nerve regeneration is implanted, and other medical agents or additives retained in or on the medical material for inducing nerve regeneration are as described above in relation to the pharmaceutical composition. The medical material for inducing nerve regeneration can also be referred to as the pharmaceutical composition for inducing nerve regeneration in the form in which a biocompatible medical material retains GFRα1 or variants thereof.

The shape of the medical material for inducing nerve regeneration is not limited, provided that it can be implanted at or near a nerve injury site, and examples include tube, sheet, and gel. The medical material for inducing nerve regeneration in tube form can be used in such a manner as to be sutured and fixed to a nerve injury site. Examples thereof include a nerve regeneration-inducing tube (Nerbridge (registered trademark)) manufactured by TOYOBO CO., LTD. and a nerve regeneration-inducing material (RENERVE (registered trademark)) manufactured by NIPRO CORPORATION. The medical material for inducing nerve regeneration in sheet form can be used in such a manner as to cover and fix a nerve injury site. The medical material for inducing nerve regeneration in gel form can be used in such a manner as to be filled in a nerve injury site or filled in a medical material for inducing nerve regeneration in tube shape. Examples of the medical material for inducing nerve regeneration in gel form include fibrin glue and alginic acid.

In one aspect, the present invention provides a method for treating nerve injury, including implanting a medical material for inducing nerve regeneration that retains an effective amount of at least one protein selected from the group consisting of GFRα1, GFRα1 mutants, and GFRα1 derivatives, to a postnatal mammalian individual in which an effective amount of GDNF is not present at or near a nerve injury site. In one aspect, the present invention also provides a method for treating nerve injury, including implanting a medical material for inducing nerve regeneration that retains an effective amount of the at least one protein and an antibody that inhibits GDNF function or a nucleic acid that suppresses GDNF expression, to a nerve injury site in a postnatal mammalian individual. In one aspect, the present invention further provides a method for treating nerve injury, including implanting a medical material for inducing nerve regeneration that retains the at least one protein at a concentration in excess of a GDNF concentration at or near a nerve injury site, to a nerve injury site in a postnatal mammalian individual.

### NCAM Stimulating Agent

The inventors of the present invention have found that GFRα1 forms a complex with NCAM and integrin α701 expressed in neurons, and that the neurite outgrowth-promoting activity of GFRα1 under a condition in which an effective amount of GDNF is not present requires a ligand for integrin α781 and is reduced by inhibition of NCAM or integrin α7β1 function. Although not bound by theory, this finding suggests that GFRα1 can stimulate NCAM and activated integrin α7β1 in a GDNF-independent manner and induce signaling for promoting neurite outgrowth (a putative molecular mechanism is illustrated on the left side of FIG. 1). Therefore, GFRα1 and variants thereof can also be used as an NCAM stimulating agent. Details regarding GFRα1 and variants thereof, preparation and use thereof, and the like are as described above in relation to the neurite outgrowth-promoting agent. The NCAM stimulating agent may also be a composition containing two or more substances.

While the present invention will be further elucidated with the following examples, it should not be considered as being limited to these examples.

### EXAMPLES

### Material and Method

### Preparation and Culture of Dorsal Root Ganglion (DRG) Neurons

Dorsal root ganglia from Lewis rats (male, 12 to 16-week-old) were each dissected into 3 to 5 pieces, which were enzymatically treated with 0.5% collagenase XI (Sigma-Aldrich) at 37° C. for 1 hour to isolate DRG neurons. The DRG neurons were seeded to achieve a concentration of 10,000 cells/cm² in a 48 well plate and cultured using DMEM/Ham's F12 with 2% B27 supplement (Thermo Fisher Scientific), 1% penicillin-streptomycin (Thermo Fisher Scientific), and 1% GlutaMax (Thermo Fisher Scientific).

### Plate Coating

To each well of a 48 well plate, 50 μg/ml of Poly-L-lysine (PLL, Sigma-Aldrich) was added, and each well was allowed to stand for 1 hour at room temperature. After the plate was washed once with PBS, 5 μg/ml of mouse EHS sarcoma basement membrane-derived Laminin (L2020, Sigma-Aldrich) was added, and the plate was allowed to stand at 37° C. for 1 day to be coated.

### Animal Experiments

Anesthetized rats were placed in the prone position, and longitudinal skin incision (4 cm long) was performed from the left buttock to the distal left thigh to expose the entire sciatic nerves. Crush injuries were created using micro-mosquito forceps (Fine Science Tools, No. 13010-12). An 8-0 Nylon thread was applied to the epineuium at the crush site for marking. Test treatments or control treatments were performed, and in some experiments, various function evaluations were performed during and after a test period. In some experiments, after a test period, the right ventricles of the rats were incised and bled under anesthesia, and the left ventricles were perfused with a PBS solution to remove blood. After transcardial perfusion and fixation with 4% paraformaldehyde-added PBS, sciatic nerves were removed.

### Sensory Function Evaluation

Starting one week before crush injury creation, the rats were acclimated by being placed in a dark room for 10 minutes each day. Before injury creation, and 4 and 8 weeks after injury creation, the rats were placed in a dark room for 5 minutes and then underwent pain stimulation to each limb with a dynamic plantar aesthesiometer (UGO BASILE). The interval between pain stimulations was 3 minutes or longer, and the same limb was not stimulated consecutively.

Heat stimulus test: Infrared heat stimulus was applied from underneath a glass chamber to the sole of each hindlimb, and the time it took for the rat to lift each hindlimb from the chamber and escape was measured three times.

von Frey test: Stimulus was applied using a 0.5-φ filament (metal) with an increase rate of 2 g/second, and the time it took for the rat to feel pain and escaped from the filament was measured three times for each hindlimb.

### Neuroelectrophysiological Function Evaluation

The anesthetized rats were placed in the prone position, and the surgical sites used for creating the crush injury were reopened from the left buttock to the distal left thigh to expose the entire sciatic nerves for measurement. Neuropack μ (NIHON KOHDEN CORPORATION) was used for the measurement. A recording needle electrode was inserted into the gastrocnemius muscle, and a 10-mV stimulus was applied using a bipolar electrode at a site slightly proximal to the crush site and another site 15 mm distal to it to generate M waves. Then, latency, amplitude, and nerve conduction velocity (MCV) were calculated. The hindlimbs on the unaffected side were used as controls. After calculation, the rats were euthanized, and the tibialis anterior muscle and gastrocnemius muscle from both hindlimbs were collected for measurement of wet muscle weights.

Immunocytochemical Staining and Immunohistochemical Staining The cultured cells were immersed in 4% PFA for 15 minutes and fixed. The sciatic nerves collected from the rats were fixed in 4% PFA at 4° C. overnight and then transferred to 30% Sucrose/PB solution and allowed to stand for 24 hours or longer. Sagittal sections of 10 μm thickness were prepared in a cryostat. The cells or sections were blocked in TBS (pH 8.4) containing 5% Normal horse serum (Thermo Fisher Scientific, Waltham, MA) and 0.25% Triton X-100 (Sigma-Aldrich, St. Louis, MO) for 1 hour, and then immersed in TBS containing the primary antibody at 4° C. ovemight and in TBS containing the secondary antibody at room temperature for 1 hour for staining. For the primary antibody, anti-pan neurofilament (pNF) antibody (mouse, 1:1000, Biolegend) was used for the cultured cells, and mouse anti-superior cervical ganglion 10 (SCG10) antibody (1:1000, Novus Biologicals) was used for the sciatic nerve tissue sections. For the secondary antibody, Alexa Fluor 594 donkey anti-mouse IgG (1:1000, Jackson Immunoresearch) was used. After washing with TBS, counterstaining was performed with DAPI.

### Image Analysis of Cultured Cells

The stained cells were observed using a fluorescence microscope BZ-X710 (KEYENCE), and the maximum length of neurites (longest neurite length) of each neuron was measured using image analysis software, Image J (Schneider et al., 2012). Cells with a longest neurite length of 50 μm or more were defined as elongating neurons, and the proportion of elongating neurons in all neurons (the percentage of elongating neurons) was calculated.

### Image Analysis of Sciatic Nerve Tissue

Three consecutive sections corresponding to the central portion of the nerve on the same slide were observed using a fluorescence microscope. Using the image analysis software, Image J, lines perpendicular to the sections were set at 10, 15, 20, and 25 mm distal and 1.5 mm proximal to the injury site, and the number of SCG10-labeled axons crossing each line was quantified. For normalization, the total number of axons in the three sections was divided by the total length of the lines to determine the axon density. The axon density at each location was divided by the axon density at an uninjured site located 1.5 mm proximal to the injury site to calculate the axon regeneration ratio. SCG10 is a marker that is specifically expressed in regenerated axons.

### Statistics

Experimental data were expressed as mean t standard error. Statistical analysis was performed using statistical analysis software JMP Pro 11.0 (SAS Institute). Student's t-test was used for comparisons between two groups. The Tukey test was used for comparisons among multiple groups. A p-value of less than 0.05 was considered significant.

### Example 1. Evaluation of Neurite Outgrowth Effect of GFRα1 on Adult Neurons

### 1-1. Neurite Outgrowth Effect of Free GFRα1

In each well of a PLL and Laminin-coated plate, a culture medium containing DRG neurons and GFRα1-Fc (rat GFRα1-Fc chimeric protein: 10 μg/ml, 560-GR, R&D Systems) or a control protein (human IgG control; 10 μg/ml, 1-001-A, R&D Systems) was added and incubated at 37° C. for 2 days (n=3). After incubation, immunocytochemical staining was performed to calculate the percentage of elongating neurons and the longest neurite length.

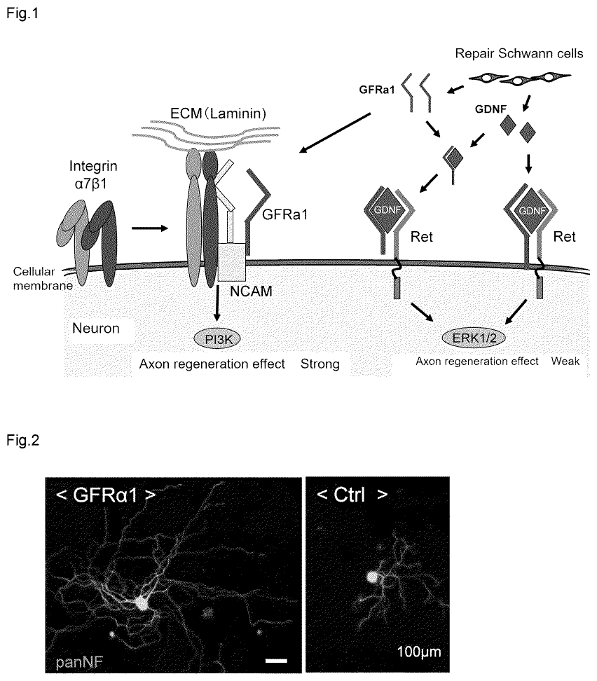

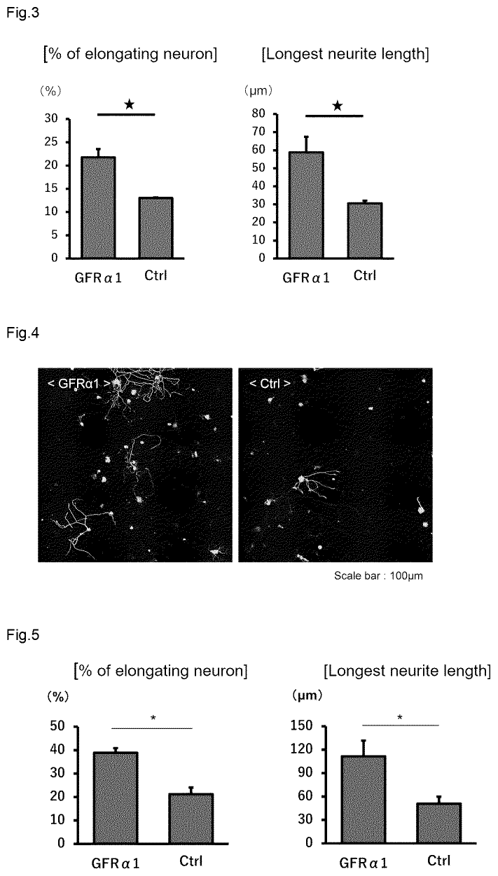

The DRG neurons cultured in the culture medium containing GFRα1-Fc exceeded the control in both of the longest neurite length and the percentage of elongating neurons, demonstrating that free GFRα1 promotes neurite outgrowth in adult neurons (FIG. 2 and FIG. 3).

### 1-2. Neurite Outgrowth Effect of Immobilized GFRα1

GFRα1-Fc (10 μg/ml) or the control protein (10 μg/ml) was added to a PLL and Laminin-coated plate, and the plate was allowed to stand at 37° C. for 1 day to be additionally coated. After the plate was washed once with PBS, DRG neurons were seeded to each well and incubated at 37° C. for 2 days (n=3). After incubation, immunocytochemical staining was performed to calculate the percentage of elongating neurons and the longest neurite length.

The DRG neurons cultured on the GFRα1-Fc-immobilized plate exceeded the control in both of the longest neurite length and the percentage of elongating neurons, demonstrating that immobilized GFRα1 promotes neurite outgrowth in adult neurons (FIG. 4 and FIG. 5).

### 1-3. Neurite Outgrowth Effect of Immobilized GFRα1 (in Relation to the Concentration of GFRα1 Added)

GFRα1-Fc 0.5 μg/ml+IgG 9.5 μg/ml, GFRα1-Fc 2.0 μg/ml+IgG 8.0 μg/ml, GFRα1-Fc 10 μg/ml, or IgG 10 μg/ml was added to a PLL and Laminin-coated plate, and the plate was allowed to stand at 37° C. for 1 day to be additionally coated. After the plate was washed once with PBS, DRG neurons were seeded to each well and incubated at 37° C. for 2 days (n=3). After incubation, immunocytochemical staining was performed to calculate the percentage of elongating neurons and the longest neurite length.

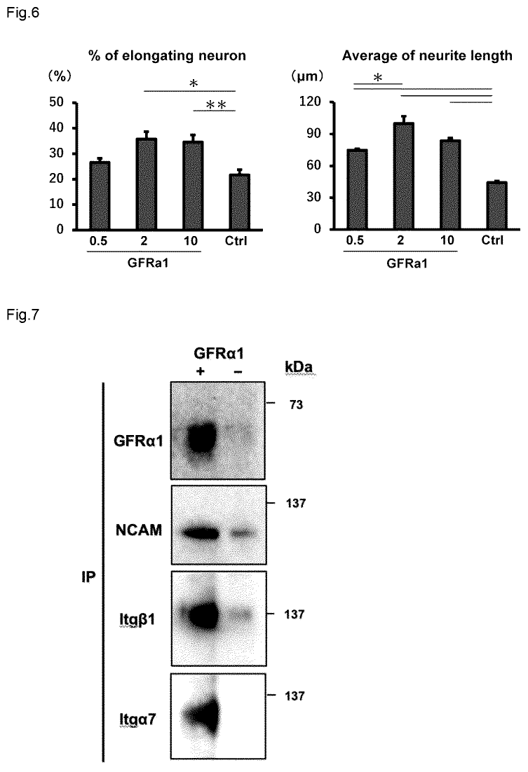

Both the longest neurite length and the percentage of elongating neurons of DRG neurons were largest on the plate coated with 2.0 μg/ml of GFRα1-Fc, demonstrating that the neurite outgrowth effect does not greatly differ even when GFRα1-Fc at a concentration higher than 2.0 μg/ml was used for coating (FIG. 6).

### Example 2. Elucidation of Mechanism of GFRα1's Effect on Neurite Outgrowth

### 2-1. Analysis of GFRα1-Binding Molecules in Neuron Lysate

GFRα1-Fc (10 μg/ml) or the control protein (10 μg/ml) was added to a PLL and Laminin-coated plate, and the plate was allowed to stand at 37° C. for 1 day to be additionally coated. After the plate was washed once with PBS, DRG neurons were seeded to each well and incubated at 37° C. for 24 hours. Utilizing the His-tag present on the used GFRα1-Fc, lysates of the cultured cells were subjected to immunoprecipitation (IP) using Dynabeads His-Tag Isolation & Pulldown (Thermo Fisher), and proteins bound to GFRα1-Fc were identified by western blotting of the resulting solutions.

The result of western blotting is illustrated in FIG. 7. The coated GFRα1-Fc was found to form a complex with NCAM and integrin α7β1 in the neurons.

### 2-2. Neurite Outgrowth Effect of Immobilized GFRα1 in Presence of NCAM Inhibitory Antibody

GFRα1-Fc (10 μg/ml) or the control protein (10 μg/ml) was added to a PLL and Laminin-coated plate and the plate was allowed to stand at 37° C. for 1 day to be additionally coated. After the plate was washed once with PBS, a culture medium containing DRG neurons and an NCAM inhibitory antibody (2 or 10 μg/ml, AB5032, Merck) or a control antibody (10 μg/ml) was added to each well and incubated at 37° C. for 48 hours (n=3). After incubation, immunocytochemical staining was performed to calculate the percentage of elongating neurons and the longest neurite length.

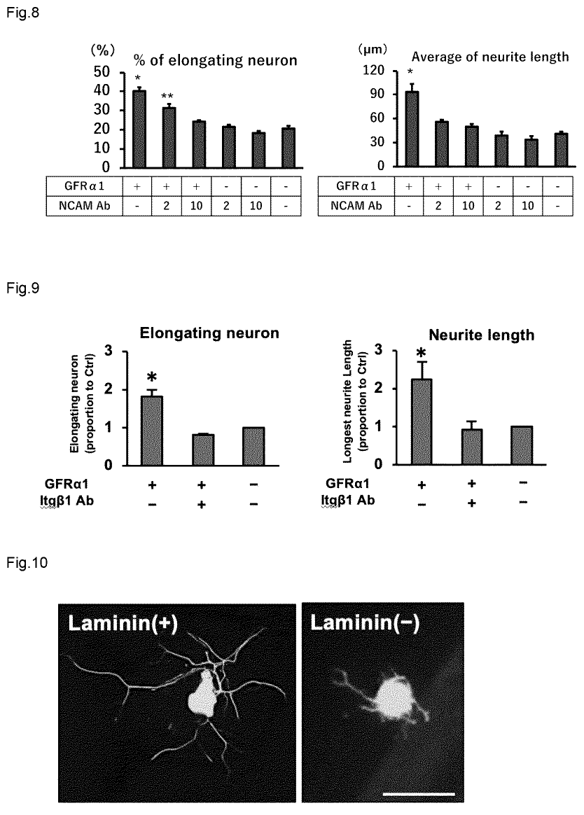

The neurite outgrowth effect of GFRα1 weakened as the concentration of the NCAM inhibitory antibody in combination increased (FIG. 8), demonstrating that the effect of GFRα1 is NCAM-dependent.

### 2-3. Neurite Outgrowth Effect of Immobilized GFRα1 in Presence of Integrin 131 Inhibitory Antibody

GFRα1-Fc (10 μg/ml) or the control protein (10 μg/ml) was added to a PLL and Laminin-coated plate and the plate was allowed to stand at 37° C. for 1 day to be additionally coated. After the plate was washed once with PBS, a culture medium containing DRG neurons and an integrin s1 inhibitory antibody (10 μg/ml, MA2910, Thermo Fisher) or a control antibody (10 μg/ml) was added to each well and incubated at 37° C. for 48 hours (n=3). After incubation, immunocytochemical staining was performed to calculate the percentage of elongating neurons and the longest neurite length.

The neurite outgrowth effect of GFRα1 almost disappeared due to the use of the integrin 31 inhibitory antibody (FIG. 9), demonstrating that the effect of GFRα1 is integrin β1-dependent.

### 2-4. Neurite Outgrowth Effect of Immobilized GFRα1 in Absence of Laminin

To each well of a 48 well plate, 50 μg/ml of Poly-L-lysine (PLL, Sigma-Aldrich) was added, and each well was allowed to stand for 1 hour at room temperature. After the plate was washed once with PBS, 5 μg/ml of Laminin (Sigma-Aldrich) or the same volume of PBS was added, and the plate was allowed to stand at 37° C. for 1 day to be additionally coated. After the plate was washed once with PBS, a culture medium containing DRG neurons was added to each well and incubated at 37° C. for 48 hours (n=3). After incubation, immunocytochemical staining was performed to calculate the percentage of elongating neurons and the longest neurite length.

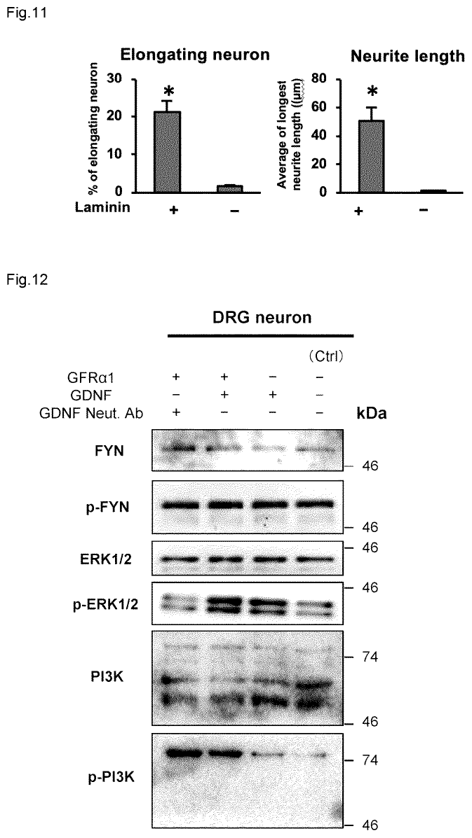

The neurite outgrowth effect of GFRα1 was not observed in the plate not coated with laminin (FIG. 10 and FIG. 11). Since laminin is a ligand for integrin α71, integrin α7R1 was presumably not activated in the absence of laminin.

### 2-5. Analysis of Intracellular Signaling by GFRα1-NCAM-Integrin α7β1 Complex

GFRα1-Fc (10 μg/ml) or the control protein (10 μg/ml) was added to a PLL and Laminin-coated plate, and the plate was allowed to stand at 37° C. for 3 hours to be additionally coated. After the plate was washed once with PBS, a culture medium containing DRG neurons, and either GDNF (recombinant rat GDNF; 2 μg/ml, 450-51, Peprotech) or the control protein (2 μg/ml), as well as either a GDNF inhibitory antibody (human/rat GDNF antibody; 10 μg/ml, AF-212-SP, R&D Systems) or a control antibody (normal goat IgG control; 10 μg/ml, AB-108-C, R&D Systems) was added to each well and incubated at 37° C. for 24 hours (n=3). Lysates of the cultured cells were subjected to immunoprecipitation (IP) using Dynabeads His-Tag Isolation & Pulldown (Thermo Fisher), and western blotting was performed on the resulting solutions to compare the expressed proteins.

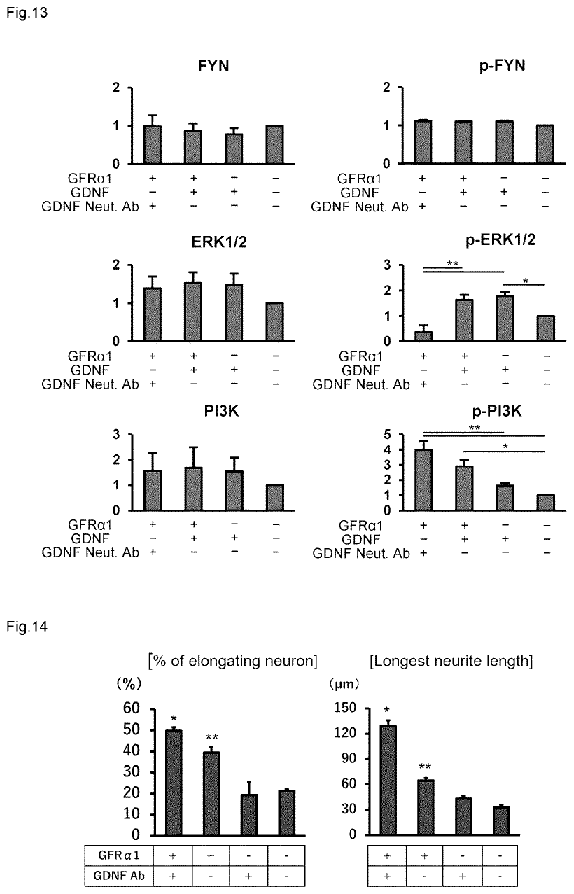

The results of western blotting are illustrated in FIG. 12, and the quantitative value for each band is illustrated in FIG. 13. The data in FIG. 13 are relative values of the intensity of the bands in each lane, corrected by the intensity of the bands in a control lane (a cell lysate cultured in a culture medium without GDNF and GDNF inhibitory antibody on the control protein-coated plate). Among the signaling molecules known to exist downstream of NCAM, FYN was not affected by GFRα1, GDNF, and the GDNF inhibitory antibody in terms of its phosphorylation. GDNF phosphorylated ERK1/2 but did not phosphorylate PI3K. When GDNF was combined with GFRα1, phosphorylation of ERK1/2 was not enhanced compared with GDNF alone, and PI3K was phosphorylated. When GFRα1 was combined with the GDNF inhibitory antibody, phosphorylation of ERK1/2 was not affected, and phosphorylation of PI3K was further enhanced, compared with when combined with GDNF. This suggests that the neurite outgrowth effect of GFRα1 in a GDNF-independent manner is mediated through phosphorylation of PI3K.

A schematic diagram of the molecular mechanism of GFRα1 hypothesized from the results of the above 2-1 to 2-5 is given in FIG. 1. The neurite outgrowth effect of GFRα1 is postulated to be mediated not only through the known signaling pathway in which GFRα1 binds to Ret via GDNF binding, activating the ERK1/2 signal (right side of FIG. 1), but also through a novel signaling pathway in which GFRα1 forms a complex with NCAM and activated integrin α7β1, activating PI3K signal (left side of FIG. 1).

### 2-6. Neurite Outgrowth Effect of Immobilized GFRα1 in Presence of GDNF Inhibitory Antibody

GFRα1-Fc (10 μg/ml) or the control protein (10 μg/ml) was added to a PLL and Laminin-coated plate, and the plate was allowed to stand at 37° C. for 1 day to be additionally coated. After the plate was washed once with PBS, a culture medium containing DRG neurons and either a GDNF inhibitory antibody (human/rat GDNF antibody; 10 μg/ml, AF-212-SP, R&D Systems) or a control antibody (normal goat IgG control; 10 μg/ml, AB-108-C, R&D Systems) was added to each well and incubated at 37° C. for 2 days (n=3). After incubation, immunocytochemical staining was performed to calculate the percentage of elongating neurons and the longest neurite length.

The combination of GFRα1 and GDNF inhibitory antibody showed stronger neurite outgrowth effect than GFRα1 alone (FIG. 14), demonstrating that the effect of GFRα1 is GDNF-independent and rather enhanced in the absence of GDNF. Based on the putative molecular mechanism illustrated in FIG. 1, it is presumed that, in GFRα1(+)GDNF inhibitory antibody(−), GDNF-GFRα1-Ret induced neurite outgrowth, whereas in GFRα1(+)GDNF inhibitory antibody(+), the formation of GDNF-GFRα1-Ret is inhibited and GFRα1-NCAM-integrin α7β1, which has stronger neurite outgrowth effect, induced neurite outgrowth.

### 2-7. Neurite Outgrowth Effect of Immobilized GFRα1 in Presence of GDNF

GFRα1-Fc (10 μg/ml) or the control protein (10 μg/ml) and either GDNF (recombinant rat GDNF; 100 ng/ml, 450-51, Peprotech) or the control protein (10 μg/ml) were added to a PLL and Laminin-coated plate, and the plate was allowed to stand at 37° C. for 1 day to be additionally coated. After the plate was washed once with PBS, DRG neurons were seeded to each well and incubated at 37° C. for 2 days (n=3). After incubation, immunocytochemical staining was performed to calculate the percentage of elongating neurons and the longest neurite length.

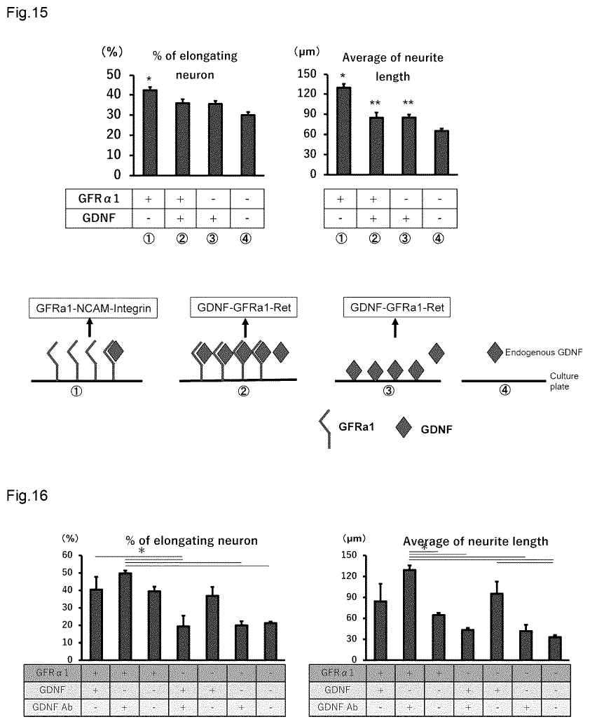

GDNF alone and the combination of GDNF and GFRα1 showed an equivalent neurite outgrowth effect, and GFRα1 alone showed stronger neurite outgrowth effect than these (the upper section of FIG. 15), demonstrating that the effect of GFRα1 is stronger than that of GDNF and attenuated in the presence of GDNF. Based on the putative molecular mechanism illustrated in FIG. 1, it is presumed that neurite outgrowth was mainly induced by GFRα1-NCAM-integrin α7β1 in GFRα1(+)GDNF(−), whereas neurite outgrowth was mainly induced by GDNF-GFRα1-Ret in GFRα1(+)GDNF(+) and GFRα1(−)GDNF(+) (the lower section of FIG. 15).

2-8. Neurite Outgrowth Effect of Immobilized GFRα1 in Presence of GDNF Inhibitory Antibody and/or GDNF

GFRα1-Fc (10 μg/ml) or the control protein (10 μg/ml) was added to a PLL and Laminin-coated plate, and the plate was allowed to stand at 37° C. for 3 hours to be additionally coated. After the plate was washed once with PBS, a culture medium containing DRG neurons, either GDNF or the control protein (100 ng/ml), and either a GDNF inhibitory antibody or a control antibody (10 μg/ml) was added to each well and incubated at 37° C. for 2 days (n=3). After incubation, immunocytochemical staining was performed to calculate the percentage of elongating neurons and the longest neurite length.

GFRα1 alone and the combination of GFRα1 and GDNF inhibitory antibody showed neurite outgrowth effect equivalent to that observed in the tests in 2-6 and 2-7 above, whereas GDNF alone and the combination of GDNF and GFRα1 showed stronger neurite outgrowth effect than that observed in the test in 2-7 above (FIG. 16). This is presumably because there was more GDNF present in the 2-8 test system than in the 2-7 test system due to the different GDNF addition method, and it bound to GFRα1-Fc on the plate and endogenous GFRα1 on the neurons to induce neurite outgrowth via GDNF-GFRα1-Ret more strongly than in the 2-7 test system.

DRG neurons, used in Examples 1 and 2, extend axons not only to peripheral tissues but also to the spinal cord and the brainstem. Therefore, the fact that GFRα1 has a neurite outgrowth effect on DRG neurons indicates that GFRα1 can exhibit neurite outgrowth effect not only on neurons in the peripheral nervous system but also neurons in the central nervous system such as the spinal cord.

### Example 3. Evaluation of Cross-Species Reactivity of GFRα1

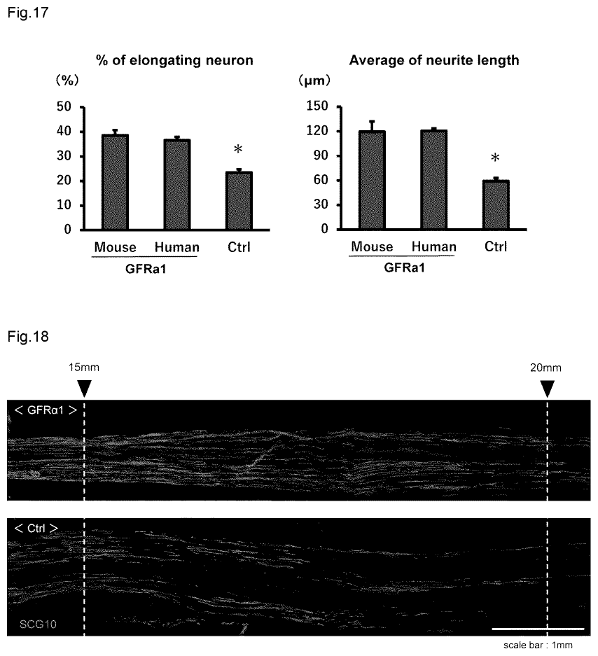

The neurite outgrowth effect was evaluated in the same manner as in the test in 1-1 of Example 1, using DRG neurons isolated from Lewis rats (8 to 14-week-old, n=3/group), except that mouse GFRα1-Fc chimeric protein (10 μg/ml, 10070-GR, R&D Systems) or human GFRα1-Fc chimeric protein (10 μg/ml, 714-GR, R&D Systems) was used instead of rat GFRα1-Fc chimeric protein (10 μg/ml, 560-GR, R&D Systems). Both mouse and human GFRα1 showed neurite outgrowth effect on the rat DRG neurons, demonstrating the cross-species reactivity of GFRα1 (FIG. 17).

### Example 4. Evaluation of Axon Regeneration Effect of GFRα1

Crush injuries were created in the left sciatic nerves of Lewis rats (8 to 14-week-old, n=3/group), and immediately thereafter, GFRα1-Fc or the control protein in 3 μg/3 μL PBS per site was administered locally at two sites 10 mm distal and 20 mm distal to the injury site. One week later, the sciatic nerves were collected from the rats, and immunohistochemical staining was performed to calculate the percentage of axon regeneration.

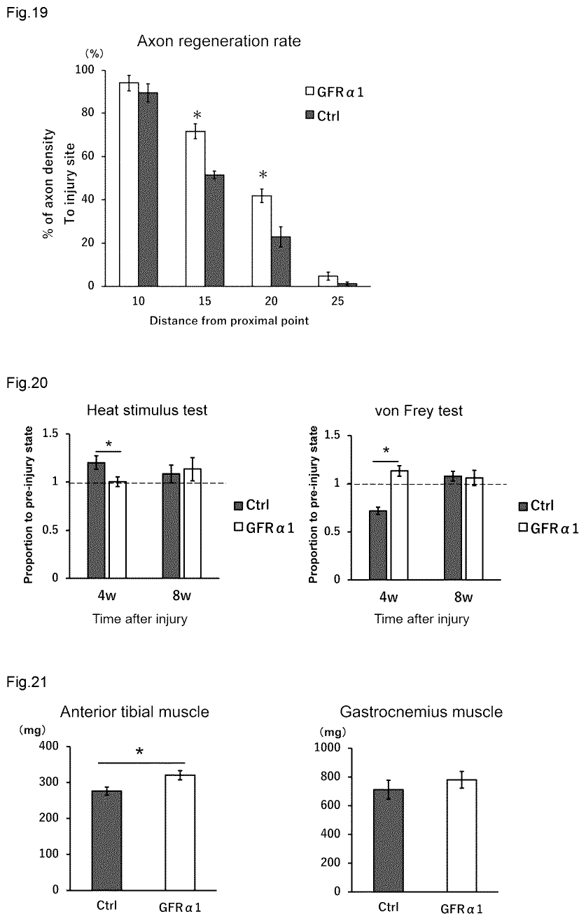

Representative immunostained images of sciatic nerve 15-20 mm distal to the injury site are illustrated in FIG. 18, and the relationship between the distance from the injury site and the percentage of axon regeneration is illustrated FIG. 19. Axon regeneration was better in the GFRα1-Fc-administered group than in the control protein-administered group, indicating that GFRα1 has axon regeneration effect. Since laminin which is an integrin α7β1 ligand is present around neurons in a living body, it is presumed that the administered GFRα1-Fc bound to GDNF secreted by Schwann cells and Ret on the neurons to induce neurite outgrowth and also formed a complex with NCAM-integrin α7β1 on the neurons to induce neurite outgrowth in a GDNF-independent manner.

### Example 5. Evaluation of Function Recovery Effect of GFRα1

Crush injuries were created in the left sciatic nerves of Lewis rats (8 to 14-week-old, n=10/group×2 groups), and immediately thereafter, GFRα1-Fc or the control protein in 3 μg/3 μL PBS per site was administered locally at two sites 5 mm distal and 15 mm distal to the injury site. One week later, GFRα1-Fc or the control protein in 3 μg/3 μL PBS per site was administered locally at two sites 10 mm distal and 20 mm distal to the injury site. The rats were maintained for 8 weeks from injury creation, and sensory function evaluation, muscle weight measurement, and neuroelectrophysiological function evaluation were performed.

Sensory Function Evaluation The response time to pain stimulation in each of the GFRα1-Fc-administered group and the control protein-administered group is illustrated in FIG. 20 as relative values to the response time to pain stimulation before crush injury creation. These relative values closer to 1 mean better sensory function. In the GFRα1-Fc-administered group, recovery of sensory function was observed 4 weeks after injury creation.

### Muscle Weight

The weights of anterior tibial muscle and gastrocnemius muscle, which are the dominant muscles of sciatic nerves, 8 weeks after injury creation are illustrated in FIG. 21. An increase in muscle weight was observed in the GFRα1-Fc-administered group compared with the control protein-administered group.

### Neuroelectrophysiological Function Evaluation

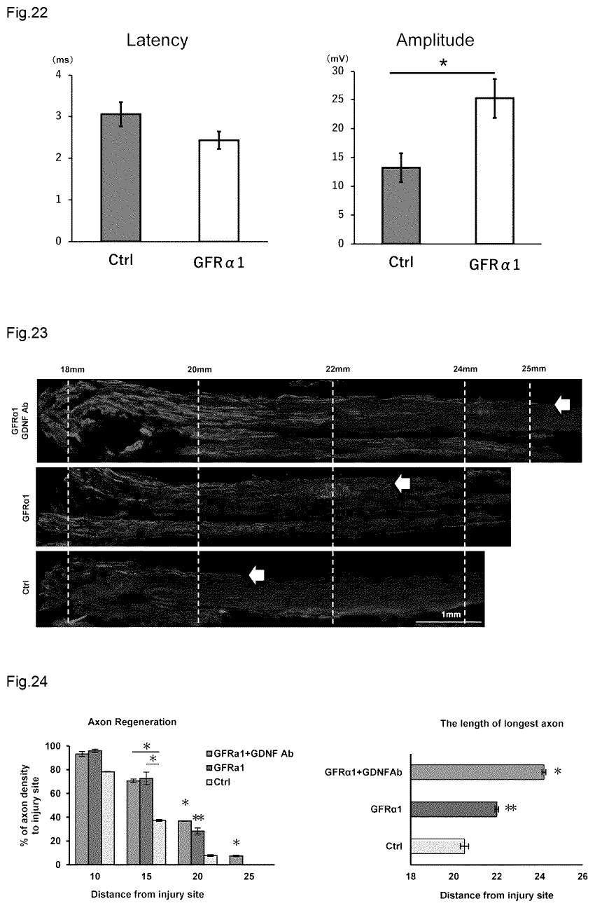

The latency, which represents the time between stimulation and muscle contraction, and the amplitude, which represents the strength of muscle contraction, are illustrated in FIG. 22. The amplitude was increased and stronger muscle contraction was observed in the GFRα1-Fc-administered group compared with the control protein-administered group, indicating that GFRα1 restores the function after sciatic nerve injury.

### Example 6. Evaluation of Axon Regeneration and Function Recovery Effects of GFRα1 in Presence of GDNF Inhibitory Antibody

(i) GFRα1-Fc 1 4 μg/4 μL PBS and a control antibody 4 μg/4 μL PBS, (ii) GFRα1-Fc 1 4 μg/4 μL PBS and a GDNF inhibitory antibody 4 μg/4 μL PBS, (iii) the control protein 4 μg/4 μL PBS and a control antibody 4 μg/4 μL PBS were prepared as test substances. Crush injuries were created in the left sciatic nerves of Lewis rats (8 to 14-week-old, n=3/group), and immediately thereafter, the above (i)-(iii) in 8 μg/8 μL PBS per site were administered locally at two sites 10 mm distal and 20 mm distal to the injury site. One week later, the sciatic nerves were collected from the rats, and immunohistochemical staining was performed to calculate the percentage of axon regeneration and the longest axon length.

Representative immunostained images of sciatic nerve 18-25 mm distal to the injury site are illustrated in FIG. 23, the relationship between the distance from the injury site and the percentage of axon regeneration is illustrated on the left side of FIG. 24, and the longest axon length is illustrated on the right side of FIG. 24. Co-administration of GFRα1 and GDNF inhibitory antibody showed higher percentage of axon regeneration and longest axon length than GFRα1 alone, indicating that the combination of GFRα1 and GDNF inhibitory antibody exhibits excellent axon regeneration effect. It is presumed that, in the rats to which GFRα1 and GDNF inhibitory antibody are co-administered, the administered GDNF inhibitory antibody bound to GDNF around the neurons to bring the surroundings of neurons into a condition in which an effective amount of GDNF is not present, and the administered GFRα1-Fc induced neurite outgrowth via GFRα1-NCAM-integrin α7β1 in a GDNF-independent manner, consequently exhibiting stronger axon regeneration effect than in the GFRα1 alone-administered rats with the presence of GDNF-GFRα1-Ret.

By administering GFRα1-Fc and a GDNF inhibitory antibody to rats in a similar manner as described above, and subsequently evaluating sensory function, muscle weight, or neuroelectrophysiological function after a certain period, the function recovery induced by the combination of GFRα1-Fc and GDNF inhibitory antibody can also be confirmed.

### Example 7. Axon Regeneration Effect of Medical Material for Inducing Nerve Regeneration That Retains GFRα1

A nerve regeneration-inducing material RENERVE (registered trademark) (NIPRO CORPORATION) was cut into a 9 mm length and immersed in rat GFRα1-Fc 10 μg/200 μL PBS or the control protein 10 μg/200 μL PBS at 37° C. overnight for coating to prepare an implant. RENERVE is a biocompatible medical material made of collagen.

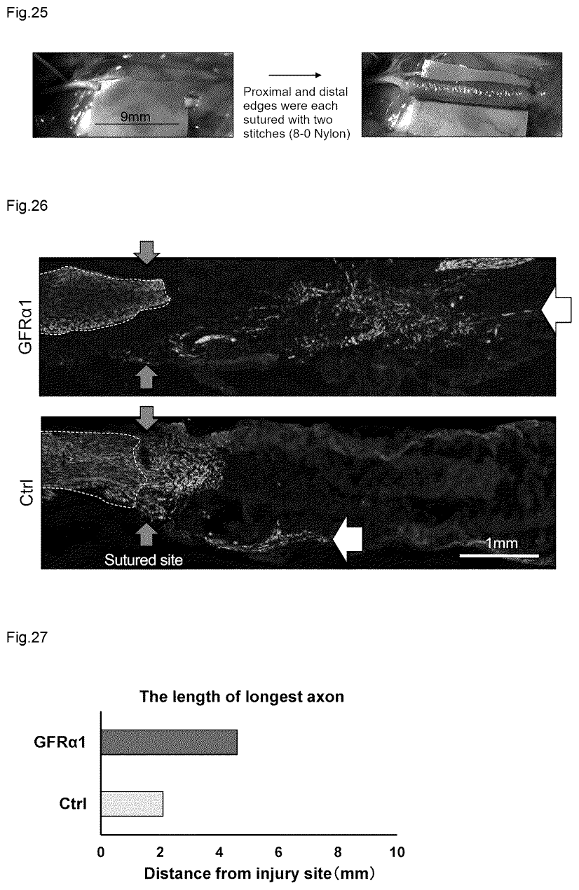

The left sciatic nerves in Lewis rats (8 to 14-week-old, n=1/group) were partially resected to create 9-mm nerve defects, and the implant coated with GFRα1-Fc or the control protein was implanted to the defects (FIG. 25). Each transected edge was sutured with two stitches using 8-0 Nylon. Two weeks later, sciatic nerves were collected from the rats, and immunohistochemical staining was performed to calculate the longest axon length.

Representative immunostained images of sciatic nerve at the implanted site are illustrated in FIG. 26, and the longest axon lengths are illustrated in FIG. 27. Longer axons were observed inside the GFRα1-Fo-coated implant, compared with the control protein-coated implant, indicating that the GFRα1-Fc-coated implant promotes axon regeneration. Since GDNF is not present in RENERVE, it is presumed that the GFRα1-Fc coated on RENERVE exclusively formed a complex with NCAM-integrin α7β1 on neurons to induce neurite outgrowth in a GDNF-independent manner and exhibited axon regeneration effect.

By implanting a GFRα1-Fc-coated implant to a nerve defect of a rat in a similar manner as described above, and subsequently evaluating sensory function, muscle weight, or neuroelectrophysiological function after a certain period, the function recovery induced by the GFRα1-Fc-coated implant can be confirmed.

### Sequence Listing Free Text

SEQ ID NO: 1 Amino acid sequence of human GFRα1 isoform a
SEQ ID NO: 2 Amino acid sequence of human GFRα1 isoform b
SEQ ID NO: 3 Amino acid sequence of human GFRα1 isoform c

1. (canceled)

2. A composition for promoting neurite outgrowth for neurons of postnatal mammalian individuals, comprising at least one protein selected from the group consisting of the following a) to d), and an antibody that inhibits glial cell-derived neurotrophic factor (GDNF) function or a nucleic acid that suppresses GDNF expression:
a) GFRα1,
b) a GFRα1 mutant having an amino acid sequence having at least 90% sequence identity with an amino acid sequence of GFRα1, and having neurite outgrowth activity,
c) a fusion protein having neurite outgrowth activity, formed by fusing GFRα1 or a GFRα1 mutant with another peptide, and
d) a chemically modified protein having neurite outgrowth activity, formed by chemically modifying at least one amino acid residue in GFRα1, a GFRα1 mutant, or a fusion protein.

3. (canceled)

4. The composition according to claim 2, wherein the amino acid sequence of GFRα1 comprises the amino acid sequence of SEQ ID NO: 1, SEQ ID NO: 2, or SEQ ID NO: 3.

5. The composition according to claim 2, wherein a carrier retains the at least one protein.

6. The composition according to claim 2, wherein the at least one protein is included in a free state.

7. The composition of according to claim 2, being a pharmaceutical composition for inducing nerve regeneration.

8-16. (canceled)

17: The composition according to claim 5, wherein the carrier is a biocompatible medical material.

18: A method for treating nerve injury, the method comprising:
administering an effective amount of at least one protein selected from the group consisting of the following a) to d):
a) GDNF family receptor al (GFRα1),
b) a GFRα1 mutant having an amino acid sequence having at least 90% sequence identity with an amino acid sequence of GFRα1, and having neurite outgrowth activity,
c) a fusion protein having neurite outgrowth activity, formed by fusing GFRα1 or a GFRα1 mutant with another peptide, and
d) a chemically modified protein having neurite outgrowth activity, formed by chemically modifying at least one amino acid residue in GFRα1, a GFRα1 mutant, or a fusion protein,
to a postnatal mammalian individual with a nerve injury in which an effective amount of GDNF is not present at or near the site of the nerve injury.

19. The method according to claim 18, wherein the amino acid sequence of GFRα1 comprises the amino acid sequence of SEQ ID NO: 1, SEQ ID NO: 2, or SEQ ID NO: 3.

20: The method according to claim 18, wherein the nerve injury is in the central nervous system.

21: The method according to claim 18, wherein the postnatal mammalian individual is an elderly individual and the nerve injury is in the peripheral nervous system.

22: The method according to claim 18, wherein an antibody that inhibits GDNF function or a nucleic acid that suppresses GDNF expression has been administered, is administered, or is to be administered to the postnatal mammalian individual, thereby an effective amount of GDNF is not present at or near the nerve injury site in the individual.

23: The method according to claim 18, wherein a carrier retains the at least one protein.

24. The method according to claim 23, wherein the carrier is a biocompatible medical material.

25: A method for promoting neurite outgrowth for neurons of postnatal mammalian individuals, the method comprising:
applying an effective amount of at least one protein selected from the group consisting of the following a) to d):
a) GDNF family receptor α1 (GFRα1),
b) a GFRα1 mutant having an amino acid sequence having at least 90% sequence identity with an amino acid sequence of GFRα1, and having neurite outgrowth activity,
c) a fusion protein having neurite outgrowth activity, formed by fusing GFRα1 or a GFRα1 mutant with another peptide, and
d) a chemically modified protein having neurite outgrowth activity, formed by chemically modifying at least one amino acid residue in GFRα1, a GFRα1 mutant, or a fusion protein,
to the neurons which are under a condition in which an effective amount of GDNF is not present.

26. The method according to claim 25, wherein the amino acid sequence of GFRα1 comprises the amino acid sequence of SEQ ID NO; 1, SEQ ID NO: 2, or SEQ ID NO: 3.

27. The method according to claim 25, wherein a carrier retains the at least one protein.

28. The method according to claim 25, wherein the at least one protein is included in a free state.


SEQUENCE LISTING (3 sequences)

SEQ ID NO: 1 - protein, 465 aa, Homo sapiens
MFLATLYFALPLLDLLLSAEVSGGDRLDCVKASDQCLKEQSCSTKYRTLRQCVAGKETNFSLASGLEAKDECRSAMEALKQKSLYNCRCKRGMKKEKNCLRIYWSMYQSLQGNDLLEDSPYEPVNSRLSDIFRVVPFISDVFQQVEHIPKGNNCLDAAKACNLDDICKKYRSAYITPCTTSVSNDVCNRRKCHKALRQFFDKVPAKHSYGMLFCSCRDIACTERRRQTIVPVCSYEEREKPNCLNLQDSCKTNYICRSRLADFFTNCQPESRSVSSCLKENYADCLLAYSGLIGTVMTPNYIDSSSLSVAPWCDCSNSGNDLEECLKFLNFFKDNTCLKNAIQAFGNGSDVTVWQPAFPVQTTTATTTTALRVKNKPLGPAGSENEIPTHVLPPCANLQAQKLKSNVSGNTHLCISNGNYEKEGLGASSHITTKSMAAPPSCGLSPLLVLVVTALSTLLSLTETS

SEQ ID NO: 2 - protein, 460 aa, Homo sapiens
MFLATLYFALPLLDLLLSAEVSGGDRLDCVKASDQCLKEQSCSTKYRTLRQCVAGKETNFSLASGLEAKDECRSAMEALKQKSLYNCRCKRGMKKEKNCLRIYWSMYQSLQGNDLLEDSPYEPVNSRLSDIFRVVPFISVEHIPKGNNCLDAAKACNLDDICKKYRSAYITPCTTSVSNDVCNRRKCHKALRQFFDKVPAKHSYGMLFCSCRDIACTERRRQTIVPVCSYEEREKPNCLNLQDSCKTNYICRSRLADFFTNCQPESRSVSSCLKENYADCLLAYSGLIGTVMTPNYIDSSSLSVAPWCDCSNSGNDLEECLKFLNFFKDNTCLKNAIQAFGNGSDVTVWQPAFPVQTTTATTTTALRVKNKPLGPAGSENEIPTHVLPPCANLQAQKLKSNVSGNTHLCISNGNYEKEGLGASSHITTKSMAAPPSCGLSPLLVLVVTALSTLLSLTETS

SEQ ID NO: 3 - protein, 344 aa, Homo sapiens
MQEGKFWKGMAETGMWGCGEWIPVEHIPKGNNCLDAAKACNLDDICKKYRSAYITPCTTSVSNDVCNRRKCHKALRQFFDKVPAKHSYGMLFCSCRDIACTERRRQTIVPVCSYEEREKPNCLNLQDSCKTNYICRSRLADFFTNCQPESRSVSSCLKENYADCLLAYSGLIGTVMTPNYIDSSSLSVAPWCDCSNSGNDLEECLKFLNFFKDNTCLKNAIQAFGNGSDVTVWQPAFPVQTTTATTTTALRVKNKPLGPAGSENEIPTHVLPPCANLQAQKLKSNVSGNTHLCISNGNYEKEGLGASSHITTKSMAAPPSCGLSPLLVLVVTALSTLLSLTETS